(19) United States
(12) Patent Application Publication
(10) Pub. No.: US 2015/0299241 A1
(43) Pub. Date: Oct. 22, 2015

(54) METHOD FOR PREPARING HIGHLY PURE DOXORUBICIN

(71) Applicant: ZHEJIANG HISUN PHARMACEUTICAL CO., LTD., Taizhou city, Zhejiang (CN)

(72) Inventors: Hua Chen, Taizhou (CN); Zhiqiang Li, Taizhou (CN); Linghui Zheng, Taizhou (CN); Shuyi Lu, Taizhou (CN); Wei Yan, Taizhou (CN); Feng Chen, Taizhou (CN); Lingping Wang, Taizhou (CN); Hua Bai, Taizhou (CN)

(73) Assignee: Zhejiang Hisun Pharmaceutical Co., Ltd., Taizhou (CN)

(21) Appl. No.: 14/646,202

(22) PCT Filed: Oct. 25, 2013
(86) PCT No.: PCT/CN2013/085989

§ 371 Date: May 20, 2015

(30) Foreign Application Priority Data
Nov. 21, 2012 (CN) 201210480803.2

(51) Int. Cl. C07H 1/06; C07H 15/252

(52) U.S. Cl. CPC C07H 1/06; C07H 15/252

(57) ABSTRACT

The present invention relates to a method for preparing highly pure doxorubicin. The method comprises the following steps: (1) chromatographing a prepurified doxorubicin solution by using macroporous resin, pre-washing the chromatographed system by using an acidic low concentration aqueous organic solvent, and performing elution by using an acidic high concentration aqueous organic solvent; (2) performing chromatographic separation on eluted components by using a preparative column, so that a highly pure doxorubicin component can be obtained, and the doxorubicin can, if needed, be separated from the aqueous solution by using conventional concentration and crystallization methods in the prior art. The present invention has the advantages such as simplicity, high yield, low cost, and less environmental pollution. The content of the prepared doxorubicin is greater than 99.5%, the content of each impurity is controlled to be lower than 0.10%, and the USP and EP standards are met.

### FIELD OF THE INVENTION

The present invention relates to a method for preparing anti-tumor antibiotics, and specifically to a method for preparing highly pure doxorubicin.

### DESCRIPTION OF THE RELATED ART

Doxorubicin is ananthracycline antibiotic (e.g. doxorubicin, daunorubicin, epirubicin, etc.) and one of the most extensively used anti-tumor drugs. It can unwind the DNA double helix, modify the template nature of DNA, block and interfere with DNA polymerase, and inhibit the synthesis of DNA and RNA, thereby blocking cell division (inhibiting nucleic acid synthesis). Doxorubicin is mainly used to treat malignant tumor diseases, such as chronic and acute leukemia, malignant lymphoma, stomach cancer, lung cancer, bladder cancer, soft tissue sarcoma, breast cancer, reticulum cell sarcoma, and malignant teratoid tumors. Its structure is shown by Formula I.

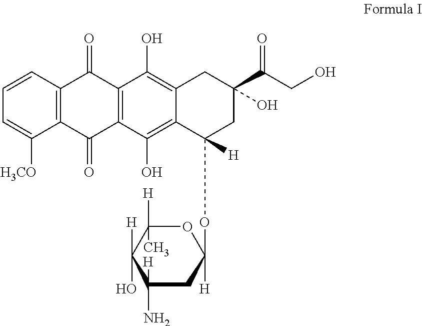

U.S. Pat. No. 3,803,124 disclosed in 1974 that doxorubicin may be prepared by a fermentation product, daunorubicin, through chemical semisynthesis. In CN1147835A, Pharmacia & Upjohn S.P.A. from Italy discloses a process for producing doxorubicin from daunorubicin using an enzyme. Currently, the method of industrial production of doxorubicin is to use daunorubicin as the intermediate through semisynthesis. However, the chemical synthesis method has issues such as inconsistent product quality, severe environmental pollution, high production cost and non-compliance with EHS requirements. CN102363755A discloses a Streptomyces sp. H323 (with the preservation number of CGMCC NO. 4827) capable of producing doxorubicin through one-step fermentation, and its doxorubicin fermentation unit satisfies the requirements for industrial production.

Currently, there are many studies on obtaining doxorubicin from semisynthesis or biological conversion with daunorubicin as the precursor and using such doxorubicin as the starting material to prepare doxorubicin through separation and purification. U.S. Pat. No. 4,861,870 uses doxorubicin obtained through semisynthesis or biological conversion as the starting material, the starting chromatographic content of doxorubicin is 70 to 80%, and a finished product of doxorubicin is obtained through adsorption with an ion exchange resin, elution with an acidic aqueous solution of an organic solvent, chromatography with a macroporous adsorption resin, elution with a slightly acidic aqueous solution of an organic solvent, and lastly crystallization. The finished product of doxorubicin prepared with this method has the chromatographic content only at 98% and the individual impurity content as high as 1.5%, which falls far short of the EP and USP standards (which require the chromatographic content to be above 99% and the individual impurity content to be lower than 0.10%), and moreover, the source of doxorubicin raw material in this process must be first subjected to semisynthesis or biological conversion, leading to a complicated process, high cost, long cycle, low yield and inapplicability for industrialization. Therefore, it is very important to find a simple purification method that is capable of preparing highly pure doxorubicin.

As pharmaceutical and biochemical industries are developing rapidly, there are increasingly extensive developments and applications of preparative liquid chromatography separation techniques, which have become important methods for isolating and purifying complex mixtures and are particularly applicable for preparing products of biological fermentation and biological conversion that have complex ingredients, plentiful byproducts and individual impurities that are difficult to isolate. The dynamic axial compression (DAC) preparation technique has advantages in many aspects, and as a result, more in-depth studies and developments have been made on this technique. The core technique of DAC is to fill a column, maintain the column pressure and empty the column through the vertical movement of a piston, a gasket with a special design is provided at the periphery of the piston to allow the piston to slide freely in the vertical direction, while maintaining a high sealing pressure. The piston movement and pressure maintenance rely on hydraulic pressure, and hydraulic force is more stable and more uniform than the spring force of an axial compression column. These techniques lead to its characteristics of low cost, long life, high column efficiency, and good symmetry and repeatability. The range of packable diameter is broad (50 mm to 1000 mm), and the separation effect is equivalent to that of an analytical column. However, no literature has been found so far regarding the separation and purification of doxorubicin products using preparative chromatography separation techniques, in particular the DAC preparation technique. Based on separation through adsorption chromatography, the present invention further employs the preparative chromatography techniques to obtain doxorubicin products with high purity that meet EP and USP standards. Moreover, the method according to the present invention has simple operations, low production cost, and high yield, which is fully applicable for industrial production.

### SUMMARY OF THE INVENTION

The object of the present invention is to provide a method for preparing highly pure doxorubicin, said method comprising the following steps:

(1) Performing chromatographic separation on a pre-purified doxorubicin solution with a macroporous adsorption resin, and collecting the doxorubicin component;

(2) Removing the organic solvent from the doxorubicin component obtained in Step (1) using a conventional method, and then performing chromatographic separation with a preparative column to obtain a highly pure doxorubicin solution; and

(3) Optionally, concentrating and crystallizing the highly pure doxorubicin solution obtained in Step (2) to prepare a doxorubicin crystal.

Wherein, with respect to the chromatography with a macroporous resin in Step (1), performing pre-washing by using an acidic low-concentration aqueous solution of an organic solvent as the pre-wash liquid, and then performing elution by using an acidic high-concentration aqueous solution of an organic solvent as the elution liquid, thereby collecting the doxorubicin component;

Wherein, with respect to the chromatography with a preparative column in Step (2), using an acidic aqueous solution of an organic solvent as the mobile phase to elute the sample, and collecting the doxorubicin-containing component in segments to obtain the highly pure doxorubicin solution;

Wherein, the pre-purified doxorubicin solution in Step (1) is prepared with the following method:

a) Using an acid to adjust the pH value of a doxorubicin fermentation liquid to acidic, filtering to obtain the pre-purified doxorubicin solution; said doxorubicin fermentation liquid may be prepared through fermentation of Streptomyces sp. H323 (with the preservation number of CGMCC NO. 4827) according to the method disclosed by CN102363755A; wherein hydrochloric acid, sulfuric acid or oxalic acid is used to adjust the pH value of the doxorubicin fermentation liquid to acidic, and the pH value is preferably 0.5 to 3.0 and more preferably 1.0 to 2.5;

or

b) Dissolving a crude product of doxorubicin in water and/or an organic solvent to obtain the pre-purified doxorubicin solution, wherein said organic solvent is selected from the group consisting of methanol, ethanol, acetone or a mixture liquid thereof; the crude product of doxorubicin may be prepared using the method of chemical semisynthesis of daunorubicin disclosed by U.S. Pat. No. 3,803,124, or prepared using the method of biological conversion of daunorubicin disclosed by CN1147835A.

Herein, the step of chromatographic separation with a macroporous adsorption resin in Step (1) comprises three processes of adsorption, pre-washing and elution.

In a preferred embodiment, the macroporous adsorption resin therein is preferably a polystyrene resin, more preferably HP20, XAD1180, XAD1600, H41, H60, CG161, HP20SS, HZ20SS, XAD-4, SP207 or SP825 resin, and more preferably HP20, HZ20SS, XAD1180 or SP207 resin.

In a preferred embodiment, the pre-washing process of the chromatographic separation with a macroporous adsorption resin therein is to perform pre-washing by using an acidic low-concentration aqueous solution of an organic solvent as the pre-wash liquid, wherein the concentration of the organic solvent in the pre-wash liquid is preferably 10 to 30% (V/V). The pre-washing process should not wash out any effective component.

In a preferred embodiment, the elution process of the chromatographic separation with a macroporous adsorption resin therein is to perform elution by using an acidic high-concentration aqueous solution of an organic solvent as the elution liquid, wherein the concentration of the organic solvent in the elution liquid is preferably 40 to 70% (V/V).

In a preferred embodiment, the organic solvent in the pre-wash liquid and the elution liquid in Step (1) preferably comprises, but is not limited to, organic solvents with medium polarity, more preferably methanol, ethanol, acetone, isopropanol or acetonitrile, and most preferably ethanol or acetone.

In a preferred embodiment, the acid in the pre-wash liquid and the elution liquid in Step (1) is preferably selected from the group consisting of hydrochloric acid, sulfuric acid, acetic acid or phosphoric acid, and the pH value of the pre-wash liquid and the elution liquid is preferably 1.5 to 4.5, and more preferably 2.0 to 3.5.

In a preferred embodiment, the preparative column used by the preparative column chromatography in Step (2) is preferably a dynamic axial compression preparative column, wherein the diameter of the dynamic axial compression preparative column is preferably 50 mm to 1000 mm, and more preferably one of the dynamic axial compression preparative column series with diameters of 50 mm, 100 mm, 200 mm, 300 mm, 500 mm, 600 mm or 800 mm.

In a preferred embodiment, the filler of the preparative column used by the preparative column chromatography therein is preferably selected from the group consisting of C18, C8, C3, polystyrenes or polymethylacrylates, and more preferably C18 or C8.

In a preferred embodiment, the particle size of the filler particles for the preparative column therein is preferably 5 μm, 10 μm or 50 μm.

In a preferred embodiment, the preparative column chromatography therein preferably uses an acidic aqueous solution of an organic solvent as the mobile phase to elute the samples, wherein the concentration of the organic solvent in the mobile phase is preferably 40 to 60% (V/V); wherein the organic solvent in the mobile phase preferably comprises, but is not limited to, organic solvents with medium polarity, more preferably methanol, ethanol, acetonitrile, isopropanol or acetone, and most preferably methanol or acetonitrile; wherein the acid in the mobile phase is preferably acetic acid, hydrochloric acid, or phosphoric acid; wherein the pH value of the mobile phase is preferably 2.5 to 3.5.

In a preferred embodiment, with respect to the preparative column chromatography in Step (2), the concentration of the doxorubicin entering the preparative column is 10 to 100 mg/mL, and preferably 50 to 80 mg/mL.

In a preferred embodiment, with respect to the preparative column chromatography in Step (2), the charging rate of the preparative column is 5 to 50 g doxorubicin/kg filler, and preferably 10 to 20 g doxorubicin/kg filler.

The present invention employs HPLC to determine the doxorubicin content and chromatographic purity with the specific method as follows:
Chromatography column: C18 column, 5 μm, 4.6×250 mm;
Mobile phase: buffer solution:acetonitrile:methanol=500:500:60;
Buffer solution: dissolve 1.44 g sodium dodecyl sulfate and 0.68 mL phosphoric acid in 500 mL ultrapure water;
Flow rate: 1.35 mL/min;
Detection wavelength: 254 mn;
Charging amount: 10 μL.

According to HPLC detection, the doxorubicin products prepared with the method of the present invention may have a chromatographic content of doxorubicin above 99.5% and an individual impurity content lower than 0.10%, and the products meet EP and USP standards.

Relative to the prior art, the present invention has the following advantages:

The doxorubicin fermentation liquid has complex ingredients, plentiful byproducts and individual impurities are difficult to isolate. The present invention first employs an adsorption chromatographic column for separation, and then employs a preparative chromatographic column for further separation and purification, and in particular, it employs the dynamic axial compression (DAC) preparation and separation technique to thoroughly solve this difficult problem. In the prior art, for example, the doxorubicin purification method disclosed by U.S. Pat. No. 4,861,870 can prepare doxorubicin with purity only around 98%, while the impurity content is up to 1.5%, which does not meet EP and USP standards. Doxorubicin prepared with the method of the present invention has a chromatographic content above 99.5% and an individual impurity content lower than 0.10%, which meets EP and USP standards. Moreover, the method according to the present invention has simple operations, low production cost, and high yield, which is very applicable for industrial production.

### BRIEF DESCRIPTION OF THE DRAWINGS

FIG. 1: HPLC chromatogram of the doxorubicin fermentation liquid in Example 1

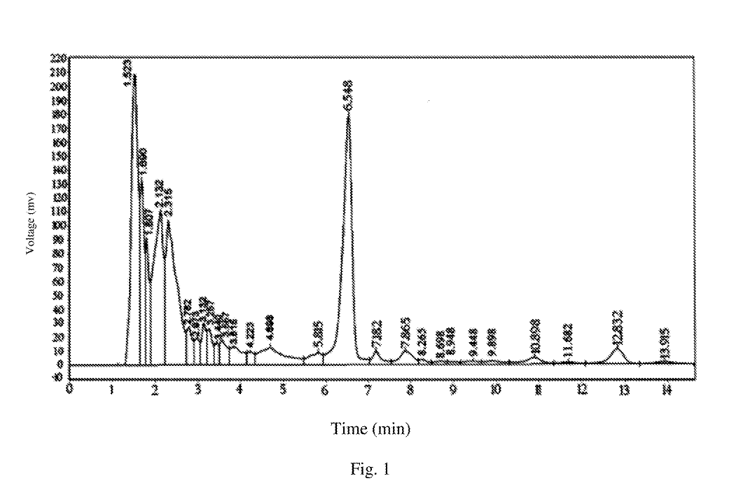

FIG. 2: HPLC chromatogram of the doxorubicin elution liquid prepared in Example 10

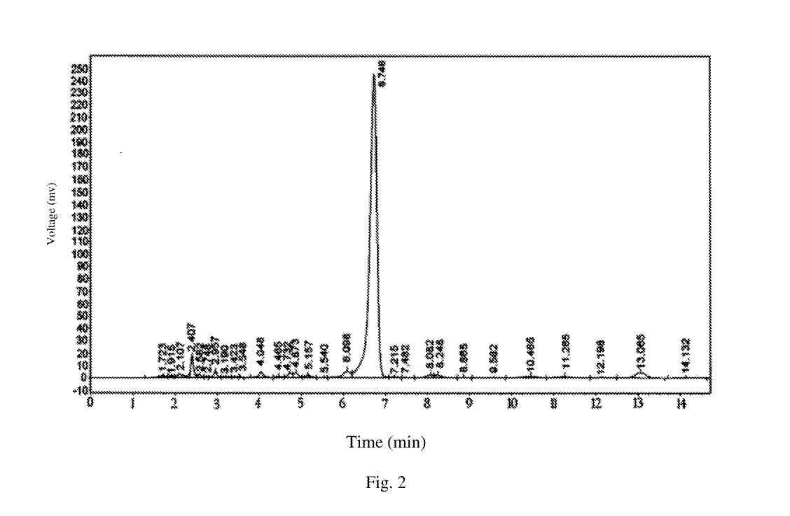

FIG. 3: HPLC chromatogram of the target component collected after doxorubicin passes through a preparative column in Example 18

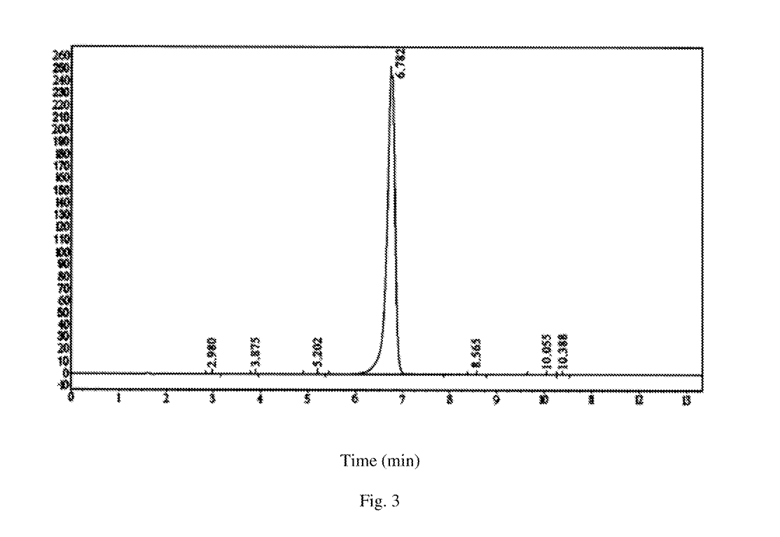

FIG. 4: ¹H NMR spectra of the highly pure doxorubicin prepared in Example 12

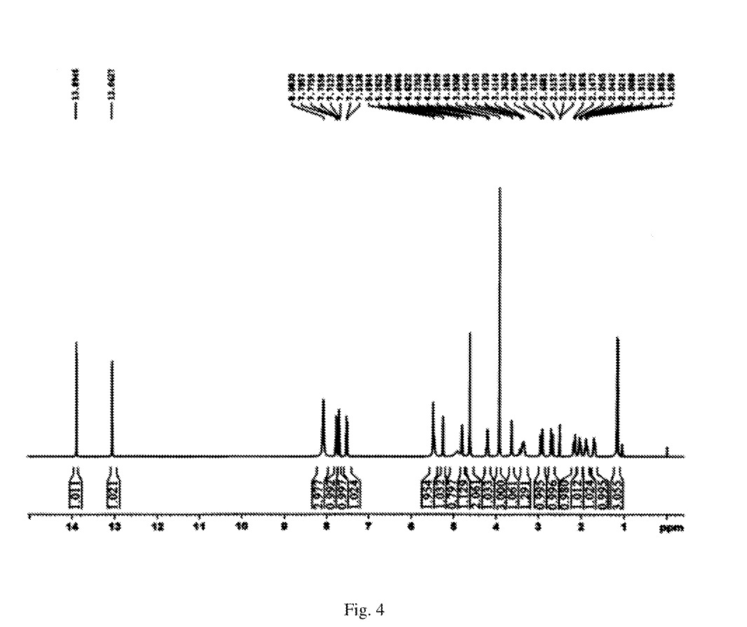

FIG. 5: ¹³C NMR spectra of the highly pure doxorubicin prepared in Example 12

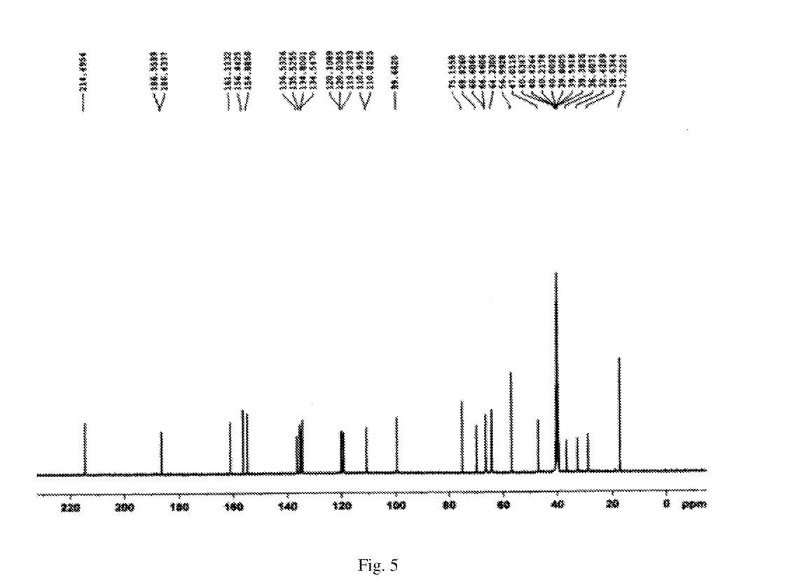

The present invention will be further described below with reference to examples. It should be understood that the preparation methods according to the examples of the present invention are only used to describe, rather than limit, the present invention. Any simple improvement made to the preparation method of the present invention without departing from the concept of the present invention shall be encompassed by the claims of the present invention.

### DETAILED DESCRIPTION OF THE PREFERRED EMBODIMENTS

### Example 1

Add 1 N hydrochloric acid to adjust the pH value of 2000 L doxorubicin fermentation liquid to 1.0 for acidification, at 3 h after acidification, filter with a plate and frame filter, obtain 1650 L of the pre-purified solution, which, upon HPLC detection, contains 1000 g doxorubicin and has a chromatographic content of 16%. Adsorb the pre-purified solution with 100 L HP20 resin, when the adsorption is completed, use 300 L 10% (V/V) aqueous solution of ethanol, and use hydrochloric acid to adjust the pH value to 2.0 as the pre-wash liquid, pre-wash, then use 400 L 50% (V/V) aqueous solution of ethanol, and use hydrochloric acid to adjust the pH value to 2.0 as the elution liquid, elute, and collect 300 L of the qualified elution liquid. The obtained elution liquid contains 800 g doxorubicin and the chromatographic content is 79%.

### Example 2

Add oxalic acid to adjust the pH value of 2000 L doxorubicin fermentation liquid to 2.5 for acidification, at 3 h after acidification, filter with a centrifuge, obtain 1700 L of the pre-purified solution, which, upon HPLC detection, contains 850 g doxorubicin and has a chromatographic content of 18%. Adsorb the pre-purified solution with 80 L XAD1180 resin, when the adsorption is completed, use 240 L 20% (V/V) aqueous solution of acetone, and use acetic acid to adjust the pH value to 3.5 as the pre-wash liquid, pre-wash, then use 320 L 40% (V/V) aqueous solution of acetone, and use acetic acid to adjust the pH value to 3.5 as the elution liquid, elute, and collect 240 L of the qualified elution liquid. The obtained elution liquid contains 680 g doxorubicin and the chromatographic content is 80%.

### Example 3

Add 1 N hydrochloric acid to adjust the pH value of 2000 L doxorubicin fermentation liquid to 3.0 for acidification, at 3 h after acidification, filter with a ceramic membrane, followed by nanofiltration to obtain 3000 L of the pre-purified solution, which, upon HPLC detection, contains 800 g doxorubicin and has a chromatographic content of 15%. Adsorb the pre-purified solution with 80 L H41 resin, when the adsorption is completed, use 240 L 30% (V/V) aqueous solution of methanol, and use sulfuric acid to adjust the pH value to 1.5 as the pre-wash liquid, pre-wash, then use 320 L 70% (V/V) aqueous solution of methanol, and use sulfuric acid to adjust the pH value to 1.5 as the elution liquid, elute, and collect 250 L of the qualified elution liquid. The obtained elution liquid contains 600 g doxorubicin and the chromatographic content is 74%.

### Example 4

Add 1 N sulfuric acid to adjust the pH value of 2000 L doxorubicin fermentation liquid to 0.5 for acidification, at 3 h after acidification, filter with a plate and frame filter, obtain 1800 L of the pre-purified solution, which, upon HPLC detection, contains 750 g doxorubicin and has a chromatographic content of 19%. Adsorb the pre-purified solution with 60 L H60 resin, when the adsorption is completed, use 180 L 25% (V/V) aqueous solution of isopropanol, and use phosphoric acid to adjust the pH value to 4.5 as the pre-wash liquid, pre-wash, then use 240 L 55% (V/V) aqueous solution of isopropanol, and use phosphoric acid to adjust the pH value to 4.5 as the elution liquid, elute, and collect 200 L of the qualified elution liquid. The obtained elution liquid contains 580 g doxorubicin and the chromatographic content is 74.5%.

### Example 5

Add oxalic acid to adjust the pH value of 2000 L doxorubicin fermentation liquid to 2.0 for acidification, at 3 h after acidification, filter with a plate and frame filter, obtain 1850 L of the pre-purified solution, which, upon HPLC detection, contains 900 g doxorubicin and has a chromatographic content of 17%. Adsorb the pre-purified solution with 80 L CG161 resin, when the adsorption is completed, use 240 L 30% (V/V) aqueous solution of acetonitrile, and use hydrochloric acid to adjust the pH value to 4.0 as the pre-wash liquid, pre-wash, then use 320 L 60% (V/V) aqueous solution of acetonitrile, and use hydrochloric acid to adjust the pH value to 4.0 as the elution liquid, elute, and collect 260 L of the qualified elution liquid. The obtained elution liquid contains 700 g doxorubicin and the chromatographic content is 76%.

### Example 6

Add 1 N hydrochloric acid to adjust the pH value of 2000 L doxorubicin fermentation liquid to 1.0 for acidification, at 3 h after acidification, filter with a plate and frame filter, obtain 1750 L of the pre-purified solution, which, upon HPLC detection, contains 880 g doxorubicin and has a chromatographic content of 20%. Adsorb the pre-purified solution with 80 L XAD1600 resin, when the adsorption is completed, use 240 L 20% (V/V) aqueous solution of acetone, and use acetic acid to adjust the pH value to 3.0 as the pre-wash liquid, pre-wash, then use 300 L 45% (V/V) aqueous solution of acetone, and use acetic acid to adjust the pH value to 3.0 as the elution liquid, elute, and collect 200 L of the qualified elution liquid. The obtained elution liquid contains 660 g doxorubicin and the chromatographic content is 76.5%.

### Example 7

Add oxalic acid to adjust the pH value of 2000 L doxorubicin fermentation liquid to 2.5 for acidification, at 3 h after acidification, filter with a centrifuge, obtain 1800 L of the pre-purified solution, which, upon HPLC detection, contains 1050 g doxorubicin and has a chromatographic content of 18%. Adsorb the pre-purified solution with 100 L XAD-4 resin, when the adsorption is completed, use 300 L 30% (V/V) aqueous solution of methanol, and use phosphoric acid to adjust the pH value to 1.8 as the pre-wash liquid, pre-wash, then use 450 L 65% (V/V) aqueous solution of methanol, and use phosphoric acid to adjust the pH value to 1.8 as the elution liquid, elute, and collect 350 L of the qualified elution liquid. The obtained elution liquid contains 790 g doxorubicin and the chromatographic content is 75%.

### Example 8

Add 1 N hydrochloric acid to adjust the pH value of 2000 L doxorubicin fermentation liquid to 3.0 for acidification, at 3 h after acidification, filter with a ceramic membrane, followed by nanofiltration to obtain 3500 L of the pre-purified solution, which, upon HPLC detection, contains 950 g doxorubicin and has a chromatographic content of 17%. Adsorb the pre-purified solution with 100 L HP20SS resin, when the adsorption is completed, use 300 L 30% (V/V) aqueous solution of isopropanol, and use acetic acid to adjust the pH value to 3.8 as the pre-wash liquid, pre-wash, then use 400 L 60% (V/V) aqueous solution of isopropanol, and use acetic acid to adjust the pH value to 3.8 as the elution liquid, elute, and collect 300 L of the qualified elution liquid. The obtained elution liquid contains 710 g doxorubicin and the chromatographic content is 77%.

### Example 9

Add 1 N sulfuric acid to adjust the pH value of 2000 L doxorubicin fermentation liquid to 0.5 for acidification, at 3 h after acidification, filter with a plate and frame filter, obtain 1600 L of the pre-purified solution, which, upon HPLC detection, contains 780 g doxorubicin and has a chromatographic content of 15%. Adsorb the pre-purified solution with 80 L SP825 resin, when the adsorption is completed, use 240 L 30% (V/V) aqueous solution of acetonitrile, and use sulfuric acid to adjust the pH value to 2.8 as the pre-wash liquid, pre-wash, then use 320 L 65% (V/V) aqueous solution of acetonitrile, and use sulfuric acid to adjust the pH value to 2.8 as the elution liquid, elute, and collect 240 L of the qualified elution liquid. The obtained elution liquid contains 585 g doxorubicin and the chromatographic content is 73%.

### Example 10

Add oxalic acid to adjust the pH value of 2000 L doxorubicin fermentation liquid to 2.0 for acidification, at 3 h after acidification, filter with a plate and frame filter, obtain 1750 L of the pre-purified solution, which, upon HPLC detection, contains 1100 g doxorubicin and has a chromatographic content of 18%. Adsorb the pre-purified solution with 100 L HZ20SS resin, when the adsorption is completed, use 300 L 25% (V/V) aqueous solution of ethanol, and use acetic acid to adjust the pH value to 2.5 as the pre-wash liquid, pre-wash, then use 400 L 55% (V/V), and use acetic acid to adjust the pH value to 2.5 as the elution liquid, elute, and collect 300 L of the qualified elution liquid. The obtained elution liquid contains 880 g doxorubicin and the chromatographic content is 81%.

### Example 11

Add 1 N hydrochloric acid to adjust the pH value of 2000 L doxorubicin fermentation liquid to 3.0 for acidification, at 3 h after acidification, filter with a ceramic membrane, followed by nanofiltration to obtain 1800 L of the pre-purified solution, which, upon HPLC detection, contains 1150 g doxorubicin and has a chromatographic content of 19%. Adsorb the pre-purified solution with 100 L SP207 resin, when the adsorption is completed, use 300 L 30% (V/V) aqueous solution of acetone, and use hydrochloric acid to adjust the pH value to 3.2 as the pre-wash liquid, pre-wash, then use 380 L 60% (V/V) aqueous solution of acetone, and use hydrochloric acid to adjust the pH value to 3.2 as the elution liquid, elute, and collect 250 L of the qualified elution liquid. The obtained elution liquid contains 920 g doxorubicin and the chromatographic content is 80%.

### Example 12

Process 2000 L fermentation liquid using the method of Example 1, obtain 250 L of the elution liquid, the elution liquid contains 700 g doxorubicin and has a chromatographic content of 79%. Depressurize and concentrate the elution liquid to obtain 14 L of a concentrated solution, the concentrated solution has a concentration of 50 mg/mL. Pass the concentrated solution through a preparative column, the model of the preparative column is DAC300, the filler is Kromasil 10 μm C18, the total column-packing quantity is 13 kg, the column-packing height is 25 cm, the single sample loading amount is 15 g/kg filler, i.e. 195 g doxorubicin (the sample charging rate is 760 mg/s, and the charging time is 4.3 min), use 60% (V/V) aqueous solution of methanol, and use acetic acid to adjust the pH value to 2.5 as the mobile phase, the elution flow rate is 2500 mL/min, and according to the sample receiving test for each syringe, the sample receiving pattern is summarized to be: starting to collect target component at 4 min. of each peak after the voltage rises to 150 mv, and ending when the voltage lowers to 100 mv, a total of 250 L of the qualified target component is collected, which, upon HPLC detection, contains 420 g doxorubicin, has the maximum individual impurity chromatographic content of 0.07%, and has the doxorubicin chromatographic content of 99.7%. Depressurize and concentrate the collected target component to 2.1 L, the concentrated solution has a concentration of 200 mg/mL, add 8.4 L acetone (4 times the volume) to stir and crystallize for 2 h, filter, and dry to obtain 402 g of highly pure doxorubicin solid.

### Example 13

Process 2000 L fermentation liquid using the method of Example 2, obtain 180 L of the elution liquid, the elution liquid contains 500 g doxorubicin and has a chromatographic content of 80%. Depressurize and concentrate the elution liquid to obtain 6.2 L of a concentrated solution, the concentrated solution has a concentration of 80 mg/mL. Pass the concentrated solution through a preparative column, the model of the preparative column is DAC200, the filler is Kromasil 10 μm C18, the total column-packing quantity is 6 kg, the column-packing height is 25 cm, the single sample loading amount is 10 g/kg filler, i.e. 60 g doxorubicin (the sample charging rate is 250 mg/s, and the charging time is 4.0 min.), use 55% (V/V) aqueous solution of acetonitrile, and use hydrochloric acid to adjust the pH value to 3.0 as the mobile phase, the elution flow rate is 1200 mL/min, and a total of 150 L of the qualified target component is collected, which, upon HPLC detection, contains 300 g doxorubicin, has the maximum individual impurity chromatographic content of 0.08%, and has the doxorubicin chromatographic content of 99.6%. Depressurize and concentrate the collected target component to 1.5 L, the concentrated solution has a concentration of 200 mg/mL, add 6 L acetone (4 times the volume) to stir and crystallize for 2 h, filter, and dry to obtain 240 g of highly pure doxorubicin solid.

### Example 14

Process 2000 L fermentation liquid using the method of Example 3, obtain 100 L of the elution liquid, the elution liquid contains 250 g doxorubicin and has a chromatographic content of 74%. Depressurize and concentrate the elution liquid to obtain 8.3 L of a concentrated solution, the concentrated solution has a concentration of 30 mg/mL. Pass the concentrated solution through a preparative column, the model of the preparative column is DAC100, the filler is Bakerbond 10 μm C18, the total column-packing quantity is 1.5 kg, the column-packing height is 25 cm, the single sample loading amount is 5 g/kg filler, i.e. 7.5 g doxorubicin (the sample charging rate is 30 mg/s, and the charging time is 4.2 min.), use 40% (V/V) aqueous solution of acetone, and use phosphoric acid to adjust the pH value to 3.5 as the mobile phase, the elution flow rate is 300 mL/min, and a total of 80 L of the qualified target component is collected, which, upon HPLC detection, contains 140 g doxorubicin, has the maximum individual impurity chromatographic content of 0.07%, and has the doxorubicin chromatographic content of 99.7%. Depressurize and concentrate the collected target component to 700 mL, the concentrated solution has a concentration of 200 mg/mL, add 2.8 L ethanol (4 times the volume) to stir and crystallize for 2 h, filter, and dry to obtain 115 g of highly pure doxorubicin solid.

### Example 15

Process 2000 L fermentation liquid using the method of Example 4, obtain 300 L of the elution liquid, the elution liquid contains 1000 g doxorubicin and has a chromatographic content of 74.5%. Depressurize and concentrate the elution liquid to obtain 10 L of a concentrated solution, the concentrated solution has a concentration of 100 mg/mL. Pass the concentrated solution through a preparative column, the model of the preparative column is DAC300, the filler is Kromasil 10 μm C8, the total column-packing quantity is 13 kg, the column-packing height is 25 cm, the single sample loading amount is 50 g/kg filler, i.e. 650 g doxorubicin (the sample charging rate is 2550 mg/s, and the charging time is 4.2 min.), use 50% (V/V) aqueous solution of ethanol, and use acetic acid to adjust the pH value to 3.0 as the mobile phase, the elution flow rate is 2500 mL/min., and a total of 200 L of the qualified target component is collected, which, upon HPLC detection, contains 550 g doxorubicin, has the maximum individual impurity chromatographic content of 0.09%, and has the doxorubicin chromatographic content of 99.5%. Depressurize and concentrate the collected target component to 2.75 L, the concentrated solution has a concentration of 200 mg/mL, add 11 L acetonitrile (4 times the volume) to stir and crystallize for 2 h, filter, and dry to obtain 500 g of highly pure doxorubicin solid.

### Example 16

Process 2000 L fermentation liquid using the method of Example 5, obtain 150 L of the elution liquid, the elution liquid contains 400 g doxorubicin and has a chromatographic content of 76%. Depressurize and concentrate the elution liquid to obtain 40 L of a concentrated solution, the concentrated solution has a concentration of 10 mg/mL. Pass the concentrated solution through a preparative column, the model of the preparative column is DAC200, the filler is Kromasil 10 μm C18, the total column-packing quantity is 6 kg, the column-packing height is 25 cm, the single sample loading amount is 20 g/kg filler, i.e. 120 g doxorubicin (the sample charging rate is 500 mg/s, and the charging time is 4.0 min.), use 50% (V/V) aqueous solution of isopropanol, and use phosphoric acid to adjust the pH value to 3.0 as the mobile phase, the elution flow rate is 1200 mL/min, and a total of 120 L of the qualified target component is collected, which, upon HPLC detection, contains 220 g doxorubicin, has the maximum individual impurity chromatographic content of 0.08%, and has the doxorubicin chromatographic content of 99.6%. Depressurize and concentrate the collected target component to 1.1 L, the concentrated solution has a concentration of 200 mg/mL, add 4.4 L methanol (4 times the volume) to stir and crystallize for 2 h, filter, and dry to obtain 200 g of highly pure doxorubicin solid.

### Example 17

Process 2000 L fermentation liquid using the method of Example 6, obtain 120 L of the elution liquid, the elution liquid contains 300 g doxorubicin and has a chromatographic content of 76.5%. Depressurize and concentrate the elution liquid to obtain 500 mL of a concentrated solution, the concentrated solution has a concentration of 60 mg/mL. Pass the concentrated solution through a preparative column, the model of the preparative column is DAC100, the filler is Bakerbond 10 μm C18, the total column-packing quantity is 1.5 kg, the column-packing height is 25 cm, the single sample loading amount is 30 g/kg filler, i.e. 45 g doxorubicin (the sample charging rate is 180 mg/s, and the charging time is 4.2 min.), use 55% (V/V) aqueous solution of acetonitrile, and use hydrochloric acid to adjust the pH value to 3.5 as the mobile phase, the elution flow rate is 300 mL/min, and a total of 100 L of the qualified target component is collected, which, upon HPLC detection, contains 165 g doxorubicin, has the maximum individual impurity chromatographic content of 0.08%, and has the doxorubicin chromatographic content of 99.6%. Depressurize and concentrate the collected target component to 825 mL, the concentrated solution has a concentration of 200 mg/mL, add 3.3 L acetone (4 times the volume) to stir and crystallize for 2 h, filter, and dry to obtain 145 g of highly pure doxorubicin solid.

### Example 18

Process 2000 L fermentation liquid using the method of Example 10, obtain 320 L of the elution liquid, the elution liquid contains 1100 g doxorubicin and has a chromatographic content of 81%. Depressurize and concentrate the elution liquid to obtain 55 L of a concentrated solution, the concentrated solution has a concentration of 20 mg/mL. Pass the concentrated solution through a preparative column, the model of the preparative column is DAC300, the filler is Kromasil 10 μm C8, the total column-packing quantity is 13 kg, the column-packing height is 25 cm, the single sample loading amount is 20 g/kg filler, i.e. 260 g doxorubicin (the sample charging rate is 1050 mg/s, and the charging time is 4.2 min.), use 45% (V/V) aqueous solution of acetone, and use hydrochloric acid to adjust the pH value to 2.5 as the mobile phase, the elution flow rate is 2500 mL/min, and a total of 300 L of the qualified target component is collected, which, upon HPLC detection, contains 880 g doxorubicin, has the maximum individual impurity chromatographic content of 0.05%, and has the doxorubicin chromatographic content of 99.8%.

### Example 19

Process 2000 L fermentation liquid using the method of Example 11, obtain 200 L of the elution liquid, the elution liquid contains 600 g doxorubicin and has a chromatographic content of 80%. Depressurize and concentrate the elution liquid to obtain 12 L of a concentrated solution, the concentrated solution has a concentration of 50 mg/mL. Pass the concentrated solution through a preparative column, the model of the preparative column is DAC200, the filler is Kromasil 10 μm C18, the total column-packing quantity is 6 kg, the column-packing height is 25 cm, the single sample loading amount is 30 g/kg filler, i.e. 180 g doxorubicin (the sample charging rate is 750 mg/s, and the charging time is 4.0 min.), use 55% (V/V) aqueous solution of methanol, and use phosphoric acid to adjust the pH value to 2.5 as the mobile phase, the elution flow rate is 1200 mL/min, and a total of 150 L of the qualified target component is collected, which, upon HPLC detection, contains 360 g doxorubicin, has the maximum individual impurity chromatographic content of 0.04%, and has the doxorubicin chromatographic content of 99.9%.

### Example 20

Dissolve 1500 g of the crude product of doxorubicin prepared using the method of chemical semisynthesis of daunorubicin disclosed by U.S. Pat. No. 3,803,124 in 100 L deionized water, obtain a pre-purified doxorubicin solution, which, upon HPLC detection, contains 1000 g doxorubicin, and has the chromatographic content of 78% (V/V).

Adsorb the pre-purified doxorubicin solution with 100 L H41 resin, when the adsorption is completed, use 300 L 30% (V/V) aqueous solution of methanol, and use hydrochloric acid to adjust the pH value to 2.5 as the pre-wash liquid, pre-wash, then use 300 L 70% (V/V) aqueous solution of methanol, and use hydrochloric acid to adjust the pH value to 2.5 as the elution liquid, elute, and collect 200 L of the qualified elution liquid. The obtained elution liquid contains 800 g doxorubicin and the chromatographic content is 97%.

Depressurize and concentrate the elution liquid to obtain 20 L of a concentrated solution, the concentrated solution has a concentration of 40 mg/mL. Pass the concentrated solution through a preparative column, the model of the preparative column is DAC300, the filler is Kromasil 10 μm C8, the total column-packing quantity is 13 kg, the column-packing height is 25 cm, the single sample loading amount is 30 g/kg filler, i.e. 290 g doxorubicin (the sample charging rate is 1100 mg/s, and the charging time is 4.4 min.), use 45% (V/V) aqueous solution of acetone, and use hydrochloric acid to adjust the pH value to 2.5 as the mobile phase, the elution flow rate is 2500 mL/min, and a total of 300 L of the qualified target component is collected, which, upon HPLC detection, contains 640 g doxorubicin, has the maximum individual impurity chromatographic content of 0.05%, and has the doxorubicin chromatographic content of 99.7%.

1. A method for preparing highly pure doxorubicin, characterized in that said method comprises the following steps:
(1) Performing chromatographic separation on a pre-purified doxorubicin solution with a macroporous adsorption resin, and collecting the doxorubicin component;
(2) Removing the organic solvent from the doxorubicin component obtained in Step (1) using a conventional method, and then performing chromatographic separation with a preparative column to obtain a highly pure doxorubicin solution; and
(3) Optionally, concentrating and crystallizing the highly pure doxorubicin solution obtained in Step (2) to prepare a doxorubicin crystal.

2. The method according to claim 1, wherein, with respect to the chromatography with a macroporous resin in Step (1), performing pre-washing by using an acidic low-concentration aqueous solution of an organic solvent as the pre-wash liquid, and then performing elution by using an acidic high-concentration aqueous solution of an organic solvent as the elution liquid, thereby collecting the doxorubicin component.

3. The method according to claim 2, wherein, with respect to the chromatography with a preparative column in Step (2), using an acidic aqueous solution of an organic solvent as the mobile phase to elute the sample, and collecting the doxorubicin-containing component in segments to obtain the highly pure doxorubicin solution.

4. The method according to claim 2, wherein the pre-purified doxorubicin solution in Step (1) is prepared with the following method:
a) Using an acid to adjust the pH value of a doxorubicin fermentation liquid to acidic, filtering to obtain the pre-purified doxorubicin solution;
or
b) Dissolving a crude product of doxorubicin in water and/or an organic solvent to obtain the pre-purified doxorubicin solution, wherein said organic solvent is selected from the group consisting of methanol, ethanol, acetone or a liquid mixture thereof.

5. The method according to claim 4, wherein the acid for adjusting the pH value in the method a) is selected from the group consisting of hydrochloric acid, sulfuric acid or oxalic acid; the pH value is 0.5 to 3.0 and preferably 1.0 to 2.5.

6. The method according to claim 1, wherein the macroporous adsorption resin in Step (1) is a polystyrene resin, preferably HP20, XAD1180, XAD1600, H41, H60, CG161, HP20SS, HZ20SS, XAD-4, SP207 or SP825 resin.

7. The method according to claim 2, wherein, in Step (1), the concentration of the organic solvent in the pre-wash liquid is 10 to 30% (V/V); the concentration of the organic solvent in the elution liquid is preferably 40 to 70% (V/V); the acid in the pre-wash liquid and the elution liquid is selected from the group consisting of hydrochloric acid, sulfuric acid, acetic acid or phosphoric acid; the pH value of the pre-wash liquid and the elution liquid is 1.5 to 4.5, and preferably 2.0 to 3.5; the organic solvent in the pre-wash liquid and the elution liquid is preferably selected from the group consisting of organic solvents with medium polarity, more preferably methanol, ethanol, acetone, isopropanol or acetonitrile.

8. The method according to claim 1, wherein, in Step (2), the preparative column used by the preparative column chromatography is a dynamic axial compression preparative column, wherein the diameter of the dynamic axial compression preparative column is 50 mm to 1000 mm, and preferably a dynamic axial compression preparative column with diameter of 50 mm, 100 mm, 200 mm, 300 mm, 500 mm, 600 mm or 800 mm; the filler of the preparative column used by the preparative column chromatography is selected from the group consisting of C18, C8, C3, polystyrenes or polymethylacrylates.

9. The method according to claim 3, wherein, in Step (2), the concentration of the organic solvent in the mobile phase is 40 to 60% (V/V); the acid in the mobile phase is selected from the group consisting of acetic acid, hydrochloric acid, or phosphoric acid; the pH value of the mobile phase is 2.5 to 3.5; the organic solvent in the mobile phase is preferably selected from the group consisting of organic solvents with medium polarity, more preferably methanol, ethanol, acetonitrile, isopropanol or acetone.

10. The method according to claim 1, wherein, with respect to the preparative column chromatography in Step (2), the concentration of the doxorubicin entering the preparative column is 10 to 100 mg/mL.

11. The method according to claim 1, wherein, with respect to the preparative column chromatography in Step (2), the charging rate of the preparative column is 5 to 50 g doxorubicin/kg filler.

12. Doxorubicin prepared with the method according to claim 1, having a chromatographic content above 99.5% and an individual impurity content lower than 0.10%.
